(19) United States
(12) Patent Application Publication
(10) Pub. No.: US 2026/0198915 A1
(43) Pub. Date: Jul. 16, 2026

(54) TISSUE CROSSING SYSTEM AND ANCHORS FOR USE THEREWITH

(71) Applicant: Transmural Systems LLC, Andover, MA (US)

(72) Inventors: Nasser Rafiee, Andover, MA (US); Morgan House, Andover, MA (US)

(21) Appl. No.: 19/201,888

(22) Filed: May 7, 2025

Related U.S. Application Data
(63) Continuation of application No. 17/086,354, filed on Oct. 31, 2020, now abandoned.
Application No. 17/086,354 is a continuation of application No. PCT/US20/45674, filed on Aug. 10, 2020.
(60) Provisional application No. 62/884,545, filed on Aug. 8, 2019.
(60) Provisional application No. 62/949,255, filed on Dec. 17, 2019.

(51) Int. Cl. A61B 17/04; A61B 17/00

(52) U.S. Cl. CPC A61B 17/0401; A61B 17/00234; A61B 2017/00243; A61B 2017/00292; A61B 2017/0409; A61B 2017/0464

(57) ABSTRACT

The present disclosure provides for novel ways of delivering tensioning elements, such as tethers, and deployable anchors, to a desired location within the anatomy of a patient. More specifically, the present disclosure provides a system and method for delivering deployable anchors to a desire location within the anatomy and tethering the anchors together through tissue. The deployable anchors may comprise anchors that have a pre-deployment elongate configuration and a post-deployment planar configuration.

### CROSS-REFERENCE TO RELATED APPLICATIONS

The present patent application claims the benefit of priority to U.S. patent application Ser. No. 17/086,354, filed Oct. 31, 2020, which in turn claims the benefit of priority to International Patent Application No. PCT/US 2020/045674, filed Aug. 10, 2020, which in turn claims the benefit of priority to Provisional Patent Application No. 62/884,545, filed Aug. 8, 2019, and U.S. Provisional Patent Application No. 62/949,255, filed Dec. 17, 2019. Each of the foregoing patent applications is hereby incorporated by reference herein in its entirety for all purposes.

### FIELD OF THE DISCLOSURE

The present disclosure relates to novel and advantageous . . . Particularly, the present disclosure relates to novel and advantageous . . . More particularly, the present disclosure relates to novel and advantageous . . .

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

In various situations, it is necessary to cross tissue in the body. For example, in treatment of structural conditions wherein an organ or luminal structure, or a portion of the organ or luminal structure needs to be reshaped or moved, it may be necessary to cross tissue of that organ or structure. For example, there are situations wherein it is desirable to reshape the structure of the heart.

Thus, there is a need in the art for systems and methods for crossing tissue. More specifically, there is a need in the art for systems and methods to anchor a tensioning element such as a tether across tissue.

### BRIEF SUMMARY OF THE DISCLOSURE

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure provides for novel ways of delivering tensioning elements, such as tethers, and deployable anchors, to a desired location within the anatomy of a patient. More specifically, the present disclosure provides a system and method for delivering deployable anchors to a desired location within the anatomy and tethering the anchors together through tissue. The deployable anchors may include anchors that have a pre-deployment elongate configuration and a post-deployment planar configuration.

While the present disclosure is directed to the delivery of such anchors and tethers in structural heart applications, the disclosed embodiments can be used for other applications such as compression of the prostate or movement of luminal structures and the like. The disclosed embodiments can be used within any organ or luminal structure that needs to be reshaped or where a portion of the organ or luminal structure needs to be moved temporarily or permanently. Thus, the disclosed embodiments are meant to be illustrative only.

In one implementation, a tissue crossing system is disclosed including a wire delivery catheter, a first wire, an anchor delivery catheter, a first anchor, and a first tether. The first wire may be configured for delivery through the wire delivery catheter. The first anchor may be configured for placement adjacent tissue and may comprise a deployable frame. The deployable frame may be configured to expand from an elongate configuration in the anchor delivery catheter pre-deployment to a planar configuration when ejected from the anchor delivery catheter. The first tether may be configured for securing to the first anchor.

In another embodiment, an anchor for deployment at a location within a body of a patient is disclosed. The anchor may have a deployable frame, a tether lumen, and a covering. The deployable frame may be configured to expand from an elongate configuration into a planar configuration having a surface area. In the elongate configuration, the deployable frame may be loaded into a catheter. In the planar configuration, the deployable frame acts to distribute force of a surface area. The covering may be provided on at least a portion of the deployable frame on a tissue adjacent surface.

In yet another embodiment, a multi-anchor system for tissue crossing is provided. The multi-anchor system may comprise a first anchor, a first tether, a second anchor, and a locking element. The first anchor may be configured for placement adjacent tissue and may comprise a deployable frame configured to expand from an elongate configuration in the anchor delivery catheter pre-deployment to a planar configuration when placed adjacent tissue. The first tether may be configured for securing to the first anchor. The second anchor may be configured for placement adjacent tissue and may comprise a deployable frame configured to expand from an elongate configuration in the anchor delivery catheter pre-deployment to a planar configuration when placed adjacent tissue.

In a further embodiment, a method for delivering and deploying a tensionable element and an anchor is provided. The method includes crossing tissue with a first crossing wire, capturing the first crossing wire, and exchanging the first crossing wire for a tensionable element. A first anchor is delivered attached to the tensionable element, wherein the first anchor expends from an elongate configuration to a planar configuration upon delivery. The method further includes crossing tissue with a second crossing wire, capturing the second crossing wire, and exchanging the second crossing wire for a tensionable element. A second anchor is delivered attached to the tensionable element, wherein the second anchor expends from an elongate configuration to a planar configuration upon delivery.

In another embodiment, an elongate catheter having a proximal end and a distal end is provided. The elongate catheter may include an elongate tubular main body and an anchor. The elongate tubular body may have a proximal end, a distal end, and at least one elongate passage therethrough, the elongate tubular main body defining a longitudinal axis along the length of the catheter. The anchor may be configured to be directed through the elongate passage, the anchor including a deployable frame configured to expand from a flattened elongate configuration into a planar configuration, the anchor being coupled to a tensionable tether.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1a illustrates an anchor with a tether extending therefrom, in accordance with one embodiment;

FIG. 1b illustrates an anchor held proximally for controlled deployment, in accordance with one embodiment;

FIG. 1c illustrates an anchor held proximally for controlled deployment, in accordance with one embodiment;

FIG. 1d illustrates an anchor in a deployed and expanded configuration, in accordance with one embodiment;

FIG. 1e illustrates an anchor in a deployed and expanded configuration, in accordance with one embodiment;

FIGS. 1f and 1g are depictions of deployable anchors in accordance with the present disclosure.

FIG. 2a illustrates a block diagram of a method for delivering a first anchor, in accordance with one embodiment;

FIG. 2b illustrates a block diagram of a method for delivering a second anchor, in accordance with one embodiment

FIG. 3 illustrates a tissue crossing system with a capture basket capturing a crossing wire, in accordance with one embodiment;

FIG. 4 illustrates a tissue crossing system with a crossing wire being exchanged for a tether, in accordance with one embodiment;

FIG. 5 illustrates a tissue crossing system with a first anchor delivered to a septum wall, in accordance with one embodiment;

FIG. 6 illustrates a tissue crossing system illustrates achieving area access and deploying a second crossing wire;

FIG. 7 illustrates a tissue crossing system with a crossing wire being exchanged for a tether, in accordance with one embodiment;

FIG. 8 illustrates a tissue crossing system with a second anchor deployed and locked, in accordance with one embodiment;

FIG. 9 illustrates a block diagram method for delivering a second anchor, in accordance with a further embodiment;

FIG. 10 illustrates a tissue crossing system with a capture basket capturing a crossing wire, in accordance with one embodiment;

FIG. 11 illustrates a tissue crossing system at introduction of a second anchor, in accordance with one embodiment;

FIG. 12 illustrates a tissue crossing system after delivering a lock, in accordance with one embodiment;

FIG. 13 illustrates a deployed tissue crossing system, in accordance with one embodiment;

FIG. 14a illustrates an anchor and lock with an anchor in a substantially planar deployed position and showing a side of the anchor that is placed away from tissue upon deployment, in accordance with one embodiment;

FIG. 14b illustrates an anchor and lock with an anchor in a substantially planar deployed position and showing a side of the anchor that is placed towards tissue upon deployment, in accordance with one embodiment;

FIG. 15a illustrates an anchor having a tether redirection feature in accordance with one embodiment;

FIG. 15b illustrates the anchor of FIG. 15a and further illustrating a tether lock, in accordance with one embodiment;

FIG. 16a illustrates an anchor having a tether redirection feature, in accordance with a further embodiment;

FIG. 16b illustrates the anchor of FIG. 16a with the tether redirection feature in a pivoted position, in accordance with one embodiment;

FIG. 17a illustrates an anchor having a frame with a honey-comb like support pattern, in accordance with one embodiment;

FIG. 17b illustrates the anchor of FIG. 17a with a material covering over the frame, in accordance with one embodiment;

FIG. 18a illustrates a wire delivery catheter, in accordance with one embodiment;

FIG. 18b illustrates a wire delivery catheter, in accordance with one embodiment;

FIG. 19 illustrates a snare and a wire extending from a wire delivery catheter, in accordance with one embodiment;

FIG. 20a illustrates a wire delivery catheter, a wire, and a protection element, in accordance with one embodiment;

FIG. 20b illustrates a protection element, in accordance with one embodiment;

FIG. 20c illustrates a protection element, in accordance with a further embodiment;

FIG. 20d illustrates a protection element, in accordance with yet a further embodiment;

FIG. 21a illustrates an anchor delivery catheter, a wire, a tether, and a protection element, in accordance with one embodiment;

FIG. 21b illustrates an attachment mechanism, in accordance with a further embodiment;

FIG. 22 illustrates deployment of a retractable anchor and tether, in accordance with one embodiment;

FIG. 23 illustrates aspects of a tissue crossing system with one anchor deployed, in accordance with one embodiment;

FIG. 24 illustrates aspects of a tissue crossing system with one anchor deployed, in accordance with one embodiment

FIG. 25 illustrates a tissue crossing system with first and second anchors deployed, in accordance with one embodiment;

FIG. 26a illustrates an anchor in a planar configuration, in accordance with a further embodiment;

FIG. 26b illustrates a view of the anchor of FIG. 26a.

FIG. 26c illustrates the anchor of FIG. 26a in an elongate configuration.

FIG. 27a illustrates an anchor in a planar configuration, in accordance with another embodiment;

FIG. 27b illustrates the anchor of FIG. 27a in a partially collapsed position; and

FIG. 27c illustrates the anchor of FIG. 27a in an elongate configuration.

### DETAILED DESCRIPTION

The present disclosure provides for novel ways of delivering tensioning elements, such as tethers, and deployable anchors, to a desired location within the anatomy of a patient. More specifically, the present disclosure provides a system and method for delivering deployable anchors to a desire location within the anatomy and tethering the anchors together through tissue. The deployable anchors may comprise anchors that have a pre-deployment elongate configuration and a post-deployment planar configuration. In the post-deployment planar configuration, the anchors act to distribute force over a surface area of the tissue.

While the present disclosure is directed to the delivery of such anchors and tethers in structural heart applications, the disclosed embodiments can be used for other applications such as compression of the prostate or movement of luminal structures and the like. The disclosed embodiments can be used within any organ or luminal structure that needs to be reshaped or where a portion of the organ or luminal structure needs to be moved temporarily or permanently. Thus, the disclosed embodiments are meant to be illustrative only.

In various embodiments, the tissue crossing system may include a wire delivery catheter, a first wire for delivery through the wire delivery catheter, an anchor delivery catheter, a first anchor for placement adjacent tissue, and a second anchor for placement adjacent tissue at a position generally opposite placement of the first anchor. A first tether may be provided for securing the first anchor and a locking element may be provided for securing the first tether to the second tether. While the term “tether” is used herein, it is to be appreciated that in all instances, “tether” refers to a tensionable element and no specific configuration is required unless specifically discussed. The anchor may have a deployable frame configured to expand from an elongate configuration pre-deployment to a planar configuration when deployed. These and other components are shown and described below.

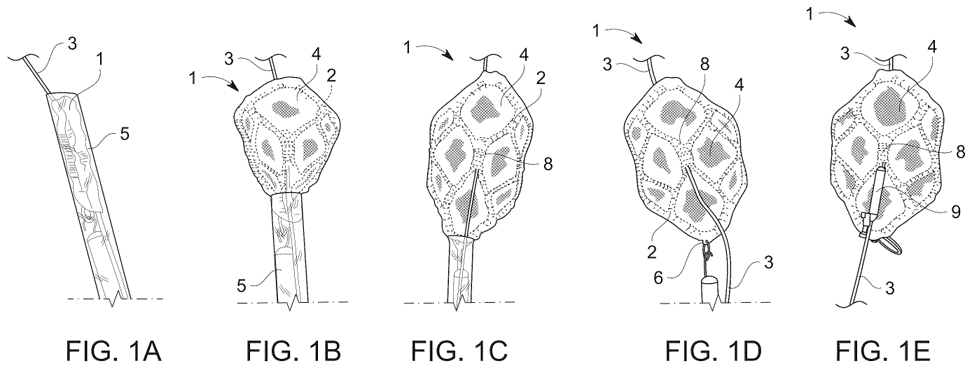

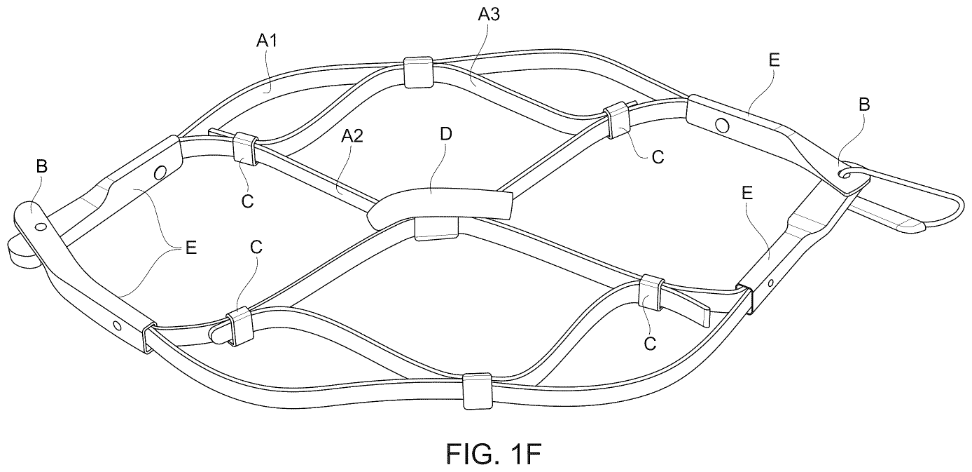

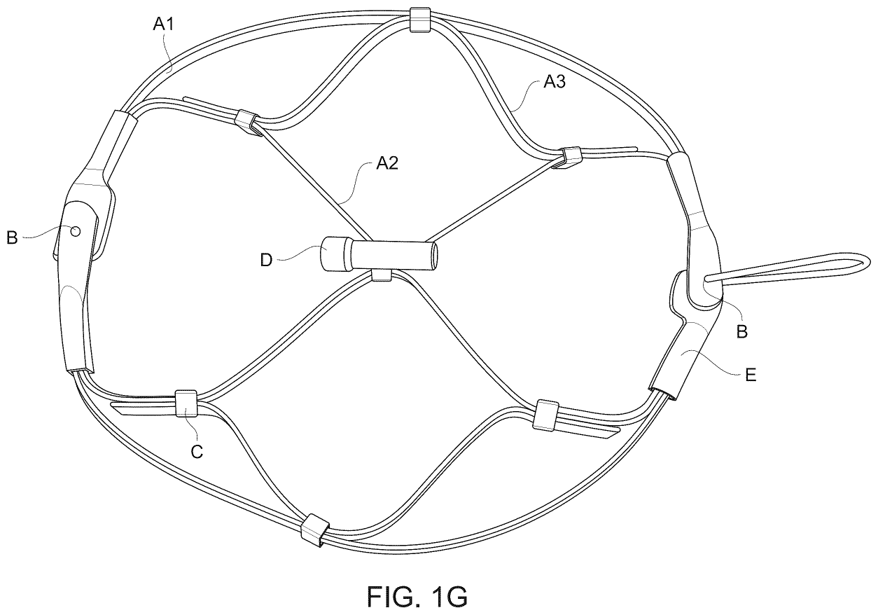

FIGS. 1a-1e illustrate an anchor 1 that may be used with a system as shown and described herein. While FIGS. 1a-1e show one specific embodiment of an anchor, it is to be appreciated that other anchor configurations that are configured to have a pre-deployment elongate configuration and a deployment substantially planar configuration may be used. FIGS. 1a-1e illustrate an anchor being deployed in a controlled manner with a proximal attachment to the anchor from the catheter.

FIG. 1a illustrates an anchor 1 with a tensionable element or tether 3 extending therefrom. The tether 3 may be connected to a second anchor or to another structure. The anchor 1 is positioned inside an anchor delivery catheter 5 or sheath. In FIG. 1a, the anchor is in a pre-deployment elongate configuration.

FIGS. 1b and 1c illustrate the anchor 1 held proximally in the anchor delivery catheter for controlled deployment. The anchor 1 may have a frame or webbing 2 that may be compressed or folded to achieve the pre-deployment elongate configuration and that expands, on its own or manually, to achieve the deployment substantially planar configuration. The frame 2 may support a material 4, such as a fabric covering, on at least one side thereof. In some embodiments, the material may be biodegradable or resorbable.

As shown, the tether 3 may be threaded through the anchor being delivered. Threading may be done through a connection point 8, which may be generally central to the frame 2. FIG. 1b illustrates the anchor 1 in a transitional configuration while FIG. 1c illustrates the anchor 1 in a substantially planar configuration. In the planar configuration, the anchor acts to distribute force over the surface area. As is described more fully below with respect to methods of placing the anchor, the tether may be delivered outside the anchor delivery catheter to aid in locking while connected to the delivery catheter.

FIG. 1d and FIG. 1e illustrate the anchor 1 fully released from the delivery catheter 5 and in an expanded configuration. In some embodiments, the anchor 1 may have a temporary holding feature to maintain position while a lock is deployed. Further, the anchor 1 may be rotated around the crossing site using a proximal attachment 6. A lock 9 may be delivered along the tether 3 to lock tension of the tether 3. In the embodiment shown in FIG. 1e, the lock is delivered along the tether 3 to the connection point 8. In alternative embodiments, the lock may be positioned at other points along the tether.

The deployable anchors, expanding from an elongate pre-deployment configuration to a planar deployed configuration, may be used in a variety of procedures. In general, they may be useful for distributing force over a surface area of tissue. Further, such anchors used with a tensionable element or tether extending therebetween can be used for any tissue crossing procedure. For illustrative purposes, a method for crossing cardiac tissue is described.

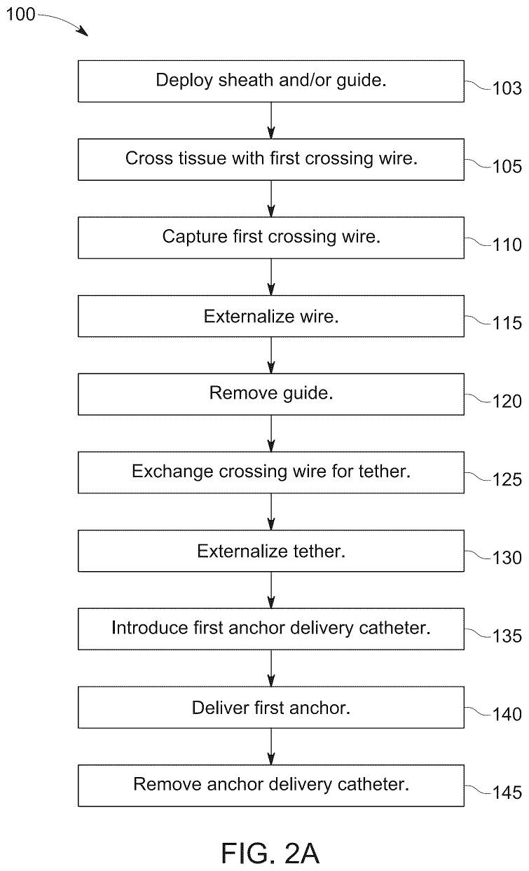

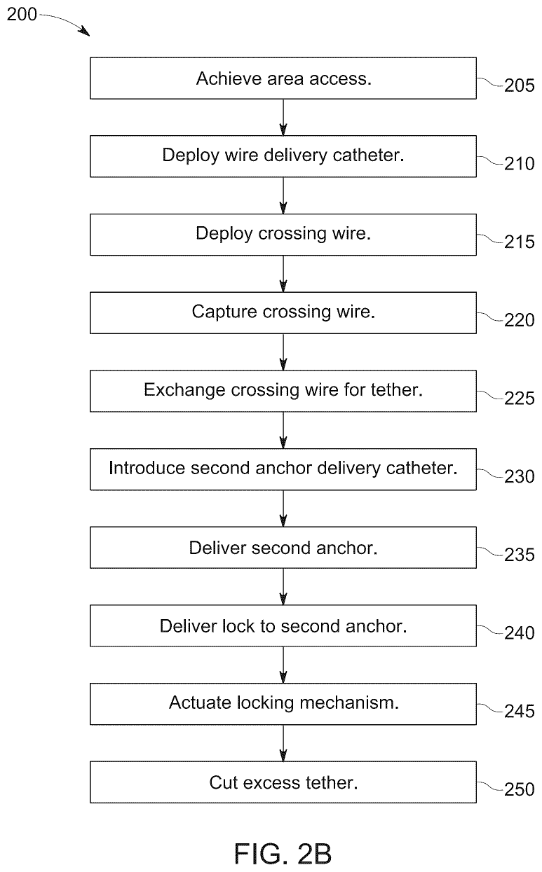

Accordingly, the tissue crossing system and anchors for use there with may be used, for example, in cardiac procedures for reshaping the heart. One method of using the system is a minimally invasive/hybrid approach shown and described with respect to FIGS. 2a-2b, and 3-8.

FIGS. 2a and 2b are block diagrams of the method for threading sutures and connecting anchors through tissue in accordance with a minimally invasive/hybrid approach embodiment. FIG. 2a illustrates a method 100 for delivery of a first anchor. FIG. 2b illustrates a method 200 for delivery of a second anchor. It is to be appreciated that the method(s) shown in FIGS. 2a and 2b are specifically described with respect to crossing heart tissue but may be used in any situation where it is necessary to cross tissue. Tissue crossing may include delivery of a first anchor and delivery of a second anchor, the anchors being tethered by a radiopaque tether across the tissue.

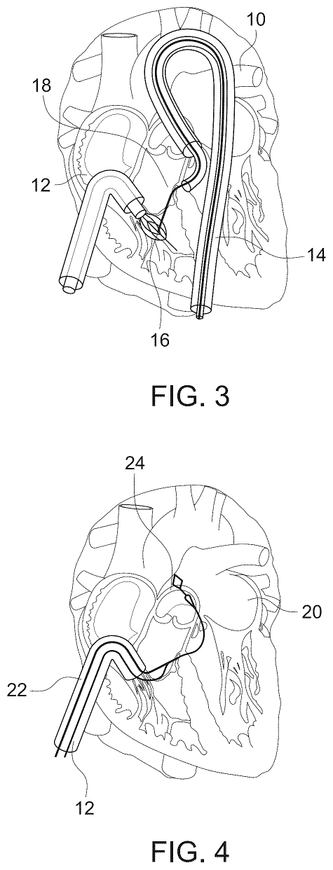

Delivery of the first or left anchor is shown and described with respect to FIG. 2 a. A sheath and/or guide is deployed 103 for receiving a first crossing wire for deployment in the body. The sheath and/or guide may be deployed through the femoral artery. The tissue is crossed with a first crossing wire 105. In a cardiac embodiment, this step 105 may comprise crossing the heart tissue at the basal anterior septum with a crossing wire. The crossing wire may be deployed through the guide or catheter deployed at 103. A capture mechanism, such as a capture basket, may then be used to capture the crossing wire 110. The capture basket may be deployed through a guide 12 extending from the femoral vein. FIG. 3 (described more fully below) illustrates the system at approximately this point in the method.

Returning to FIG. 2a, after capture of the first crossing wire, the first crossing wire is externalized 115. Optionally, the tissue may be protected at the crossing point using a soft tipped catheter. One or more of the guides or sheaths may be removed 120. In some embodiments, removal of the guides or sheaths may comprise removing a guide from the femoral artery.

Next, the first crossing wire is exchanged for a tensionable element or tether 125. In some embodiments the tether may be radiopaque. FIG. 4 (described more fully below) illustrates the system at this stage. After the crossing wire is exchanged for a tether, the tether may be externalized 130. In some embodiments, the tether may be externalized out of the femoral vein.

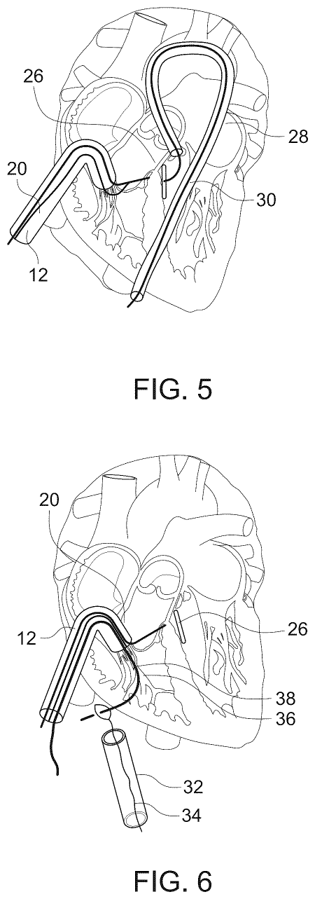

A first anchor delivery catheter is introduced 135. In a cardiac embodiment, this may comprise introducing a first or left delivery catheter through the femoral artery. A first anchor may then be delivered to a desired location through the anchor delivery catheter 140. When in the anchor delivery catheter, the anchor is in a pre-deployment elongate configuration. Upon delivery, the anchor is expanded to a post-deployment planar configuration. This may happen automatically upon expulsion from the catheter or may be done manually. In the embodiment shown in FIG. 5 (described more fully below), a left anchor is delivered to the septum wall. At this point, delivery of the first or left anchor is completed and the first anchor delivery catheter may be removed 145.

One embodiment of delivery of the second or right anchor 200 is shown and described with respect to FIG. 2b. In this embodiment, delivery of the second anchor may be done after delivery of the first anchor.

Area access for delivery of the second anchor is achieved 205. This may be done by insufflating the area and performing a mini-thoracotomy. For example, insufflation with carbon dioxide may be done through the right atrial appendage and a mini-thoracotomy may be performed to gain subxiphoid axis with a subxiphoid sheath.

A wire delivery catheter is deployed 210. Deployment of the wire delivery catheter 210 may be through the femoral vein and inferior vena cava (IVC). The wire delivery catheter may be an articulating catheter and may be positioned in the right ventricle facing free wall. A crossing wire is deployed through the wire delivery catheter 215. The crossing wire may be, for example, an electrified crossing wire. The crossing wire is captured 220. A capturing snare may be used for capturing the electrified crossing wire. In some embodiments, the capturing snare may be deployed through the subxiphoid sheath. The position of the system at this point is shown in FIG. 6.

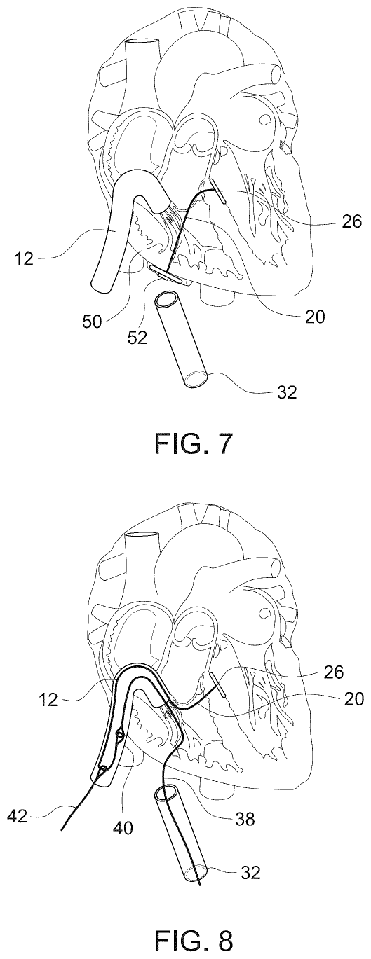

The crossing wire is exchanged for a radiopaque tether 225. This may be done through the free wall. In some embodiments, the free ends of the radiopaque tether and the crossing wire are coupled, such as by crimping, prior to exchange. FIG. 7 (described more fully below) illustrates exchange of the crossing wire for the radiopaque tether.

A second anchor delivery catheter is introduced 230. The second anchor delivery catheter may be introduced through the subxiphoid sheath and articulated towards the free wall or right ventricle. The second anchor is delivered 235. When in the anchor delivery catheter, the anchor is in a pre-deployment elongate configuration. Upon delivery, the anchor is expanded to a post-deployment planar configuration. This may happen automatically upon expulsion from the catheter or may be done manually. In a cardiac embodiment, the second anchor may be a right anchor and may be delivered on the free wall. During and after delivery, tension may be maintained on the radiopaque tether. A lock may be delivered to the second anchor 240. A locking mechanism of the lock is actuated 245 to fix the tension of the radiopaque tether. Such actuation 245 may be done when applied tension on the tether is satisfactory. Excess tether 250 is cut. FIG. 8 (descripted more fully blow) illustrates the system at this point in the method.

It is to be appreciated that the wire delivery catheter, the crossing wire, the guide, and the subxiphoid sheath are removed as makes sense during and/or after the procedure such that only the first anchor, the tether, the second anchor, and the lock remain in place.

Now turning to depiction of the system as shown in FIGS. 3-8, crossing of heart tissue may be done at the basal anterior septum as described in FIGS. 2a and 2b. The tissue crossing system and anchor achieve such crossing while avoiding the anchor affecting the aortic valve. FIG. 1 illustrates early stages of deployment of the tissue crossing system. As shown guides 10, 12 are deployed through the femoral artery and vein, respectively, to the heart. The exact configuration of the guides may vary. In one embodiment, the guides may be 14 F guides deployed through 16 F sheaths. A wire delivery catheter is deployed through the first guide 10 extending from the femoral artery. A capture basket 16 is deployed through the second guide 12 extending from the femoral vein. A first crossing wire 18 is deployed through the wire delivery catheter 14. The capture basket 16 may be used to snare the first crossing wire 18. In some embodiments, the crossing wire may be electrified.

As shown in FIG. 4, exchanging the crossing wire 18 for a tether 20 may include inserting a protection catheter 22 through the guide 12 in the femoral vein. A connector 24, such as a crimp connector, is advanced through the protection catheter 22. In one embodiment, the connector is 24 crimped onto the crossing wire 18 to achieve connection. The tether 20 and left anchor (see FIG. 5) may then be advanced.

FIG. 5 illustrates delivery of a first anchor, in accordance with one embodiment. More specifically, FIG. 5 illustrates an anchor 26 delivered to the septum wall, in accordance with one embodiment. The anchor 26 is referred to herein as a first anchor or a left anchor but it is to be appreciated such reference is intended for illustrative purposes only. An anchor delivery catheter 28, referred to as a left anchor delivery catheter, may be introduced through the femoral artery and the left anchor 26 deployed to the septum wall therethrough by pulling the radiopaque tether 20 to which the anchor 26 is attached through guide 12. An anchor retaining suture 30 may be used to secure the left anchor 26 in position. The anchor delivery catheter 28 may be removed. The free end of the tether 20, through the femoral vein, may be secured to maintain tension on the system.

FIG. 6 illustrates achieving area access and deploying a second crossing wire, in accordance with one embodiment. As described with respect to FIG. 2a, a mini-thoracotomy may be performed to gain subxiphoid access. FIG. 6 illustrates the subxiphoid sheath 32. A snare 34 can be deployed through the subxiphoid sheath 32. The snare 34 may be, for example, a capture basket or goose-neck type snare. Using a wire delivery catheter 36 through the sheath 12, a crossing wire 38, which may be referred to as a second crossing wire, is advanced through the femoral vein access and inferior vena cava. The crossing wire 38 may be, for example, a 0.014″ electrified crossing wire. The wire delivery catheter 36 may be a position articulating catheter. In the embodiment shown, the wire delivery catheter 36 is positioned in the right ventricle facing free wall. The second crossing wire 38 is positioned to traverse through the free wall and snare through the subxiphoid axis. At this stage, the radiopaque tether 20 still extends from the guide 12 to the anchor 26. A capturing snare 34 may be deployed through the subxiphoid sheath 32 for capturing the electrified crossing wire 38. The snare 34 may be, for example, a capture basket or goose-neck type snare.

FIG. 7 illustrates exchange of the electrified crossing wire 38 for the radiopaque tether 20. This may be done through the free wall. In some embodiments, the free ends of the radiopaque tether 20 and the crossing wire 38 are coupled, such as by crimping, prior to exchange. A connector 40 is shown connecting the crossing wire 38 and the radiopaque tether 20. A straightening snare 42 or catheter may be used to substantially prevent the radiopaque tether 20 and wire 38 from tangling.

FIG. 8 illustrates the system after positioning of the second anchor and locking of the anchor. The first anchor 26 is positioned on the septum wall. The second anchor 50 is positioned on the free wall. A tether 20, for example a radiopaque tether, extends between the first anchor 26 and the second anchor 50. A lock 52 locks the tether 20 at a desired tension. At this point, the guide 12 and the subxiphoid sheath 32 may be removed.

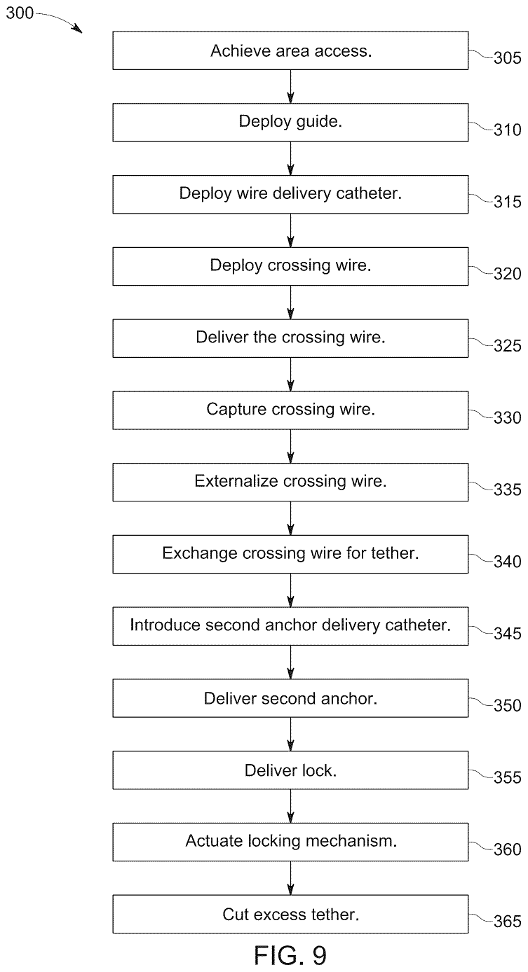

Another method of using the system uses a fully percutaneous approach for delivery of the right anchor. This approach is shown and described with respect to FIGS. 9-13. It is to be appreciated that this method specifically focuses on delivery of a right anchor in a cardiac embodiment. Accordingly, the method shown and described with respect to FIGS. 2a and 3-5 may be used for delivery of a left anchor prior to commencement of the method shown and described with respect to FIGS. 9-13.

FIG. 9 is a block diagram of a method for threading sutures and connecting anchors through tissue using a fully percutaneous approach for delivery of a right anchor in a cardiac embodiment. Tissue crossing may include delivery of a first anchor and delivery of a second anchor, the anchors being tethered by a radiopaque across the tissue.

Delivery of the first or left anchor is shown and described with respect to FIG. 2a-5. The method shown and described with respect to FIG. 9 thus may commence when the system is generally in the configuration shown in FIG. 5. More specifically, the method may be initiated when the left anchor 26 is in place at the septum wall.

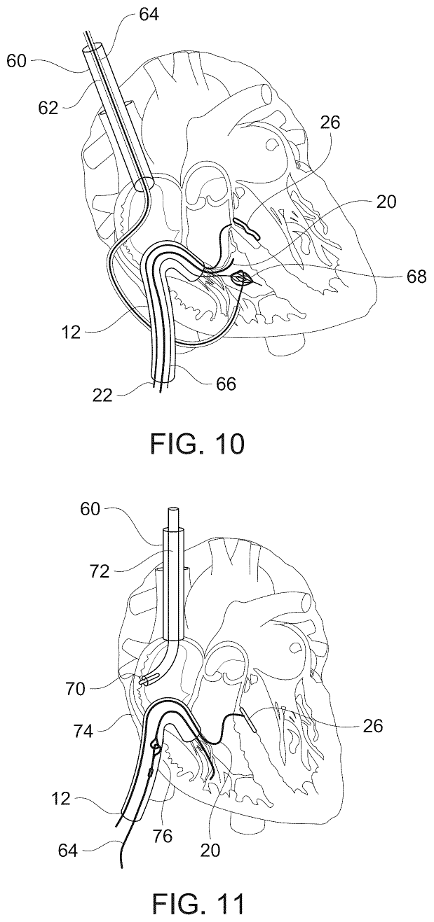

Area access for delivery of the second anchor is achieved 305. This may be done by insufflating the area and is generally for native pericardium only. A guide is then deployed 310 and a wire delivery catheter deployed 315 therethrough. A crossing wire is delivered 320 through the right atrial appendage into the pericardial space using the wire delivery catheter and jugular guide. The crossing wire is delivered to its desired position 325. This may comprise traversing the wire delivery catheter and the crossing wire through the pericardial space towards the apex. The wire delivery catheter may be articulated towards the right ventricle and the electrified crossing wire delivered through the free wall into the right ventricle. The crossing wire is captured 330. This may comprise snaring the crossing wire into the wire delivery catheter or the guide using a wire capture basket. The crossing wire is externalized 335. FIG. 10, described more fully below, illustrates the system at this stage in the method.

The crossing wire is exchange for a tether 340. This may comprise crimping free ends of a tether, such as a radiopaque tether, and the crossing wire together using a crimp connector. Exchanging of the crossing wire for the tether may be done through the free wall and out a femoral venous access sheath.

A second anchor delivery catheter is introduced 345. This may be done through jugular venous access. The second anchor is delivered 350. This may comprise delivering the right atrial appendage into the pericardial space and traversing it through the pericardial space until it is deployed on to the free wall. Tension may be maintained on the radiopaque tether. FIG. 11, described more fully below, illustrates the system after introduction of the second, or right, anchor but before full deployment of the second anchor.

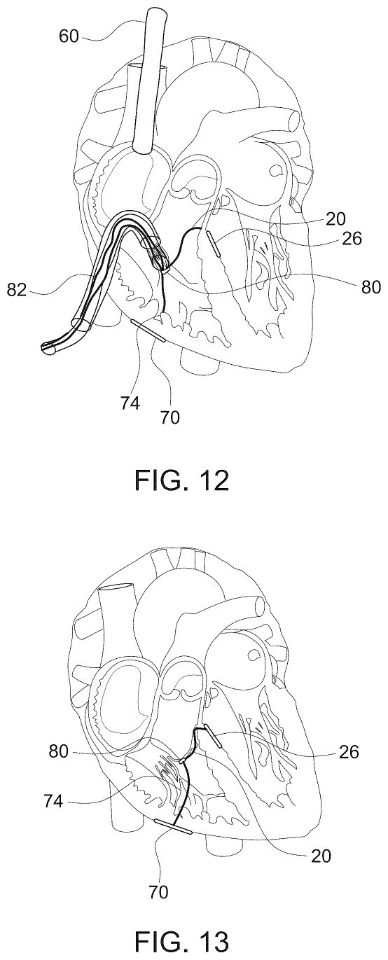

A lock is delivered 355. In some embodiments, this may comprise delivering a lock over both first and second radiopaque tethers, the first extending from the first anchor and the second extending from the second anchor. Delivery of the lock 355 may be done using a lock delivery catheter. FIG. 12 illustrates the system after the lock is deployed. The locking mechanism is actuated 360. In some embodiments, the tethers may be pulled to achieve a satisfactory tension before the locking mechanism is actuated. The excess tether is cut 365, such as by using a tether cutter. FIG. 13 illustrates the fully deployed system.

Now turning to illustration of the system during the method shown in FIG. 9. FIG. 10 illustrates the system at initial stages of delivering a right anchor using a fully percutaneous approach. As shown, a left anchor 26 is deployed to the septum wall. A tether 20 extends from the anchor 26 into a catheter 22. The catheter 22 and tether 20 in turn extend into a guide 12. The guide 12 may be a femoral venous access sheath.

A guide 60, optionally a 14 F guide, also referred to as a jugular sheath, is deployed in the jugular vein. A wire delivery catheter 62 is inserted through the guide 60 and a crossing wire 64 is deployed through the wire delivery catheter 62. The crossing wire 64 may be a 0.014″ electrified guidewire in some embodiments. The wire delivery catheter 62 and crossing wire 64 exit the right atrial appendage into the pericardial space. The crossing wire crosses the free wall. A wire capture snare 66 and capture basket 68 are delivered through the guide 12. The capture basket 68 captures the crossing wire after the crossing wire has crossed the free wall.

FIG. 11 illustrates the system at introduction of the second, or right, anchor. The first, or left, anchor 26 is in place at the septum wall with a first tether 20 extending therefrom. A right anchor delivery catheter 72 is extended through the jugular sheath 60. A second anchor 70 is delivered through the anchor delivery catheter 72. A tether 74, such as a radiopaque tether extends from the second anchor 70 to the crossing wire 64. The ether 74 traverses through the pericardial space. A connector 76, such as a crimp connector, connects the tether 74 to the crossing wire 64. The crossing wire thus may traverse the pericardial space and be exchanged for the tether to deploy the second anchor 70 onto the free wall.

FIG. 12 illustrates the system after the lock is delivered at step 355 of FIG. 9. The first, or left, anchor 26 is in place at the septum wall with a first tether 20 extending therefrom. The second, or right, anchor 70 is in place on the free wall with a second tether 74 extending therefrom. In the embodiment shown, both the first tether 20 and the second tether 74 extend from the respective anchors into the femoral access sheath 12. The lock 80 couples the first tether 20 and the second tether 74. The lock 80 as associated with the first tether 20 and the second tether 74. For example, the lock 80 may be slid over the first tether 20 and the second tether 74 and delivered to the desired location. Delivery of the lock 80 may be done using a lock delivery catheter 80. In some embodiments, the lock delivery catheter 80 may be extended through the femoral venous access sheath 12.

FIG. 13 illustrates the fully deployed system. As shown, the first, or left, anchor 26 is in place at the septum wall and the second, or right, anchor 70 is in place on the free wall. A first tether 20 extends from the first anchor 26 and a second tether 74 extends from the second anchor 70. The first tether 20 and the second tether 74 are tensioned and locked together with a lock 80.

Components of the system will now be more specifically shown and described. In general, an anchor may be deployed through an anchor delivery catheter. The anchor delivery catheter may be an elongate catheter having a proximal end and a distal end. The elongate catheter may include an elongate tubular main body and an anchor. The elongate tubular body may have a proximal end, a distal end, and at least one elongate passage therethrough, the elongate tubular main body defining a longitudinal axis along the length of the catheter. The anchor may be configured to be directed through the elongate passage. The anchor may include a deployable frame configured to expand from a flattened elongate configuration into a planar configuration, the anchor being coupled to a tensionable tether. In a planar configuration, the deployable frame acts to distribute force over the surface area.

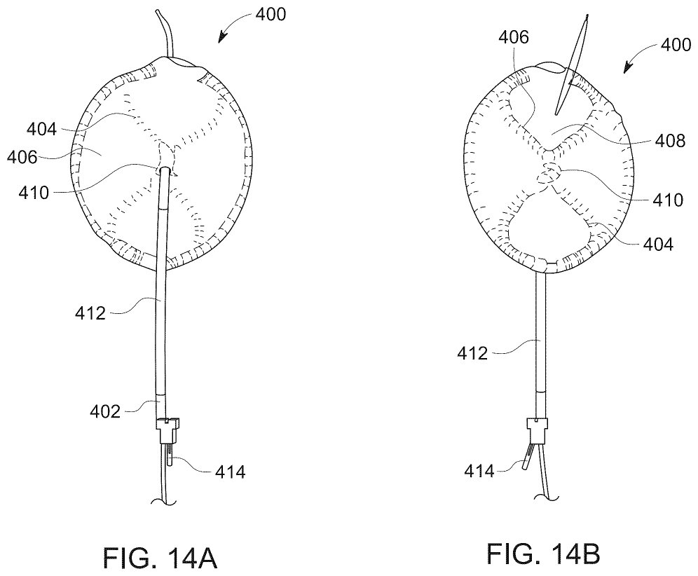

FIGS. 14a and 14b illustrate an anchor 400 and lock 402 with the anchor 400 in a substantially planar deployed configuration. FIG. 14a illustrates a side of the anchor 400 that may be placed away from tissue upon deployment. FIG. 14b illustrates a side of the anchor 400 that may be placed towards tissue upon deployment. In this embodiment, the anchor 400 may be delivered with the lock 402 attached rather than delivered separately. In some embodiments, the lock 402 may be referred to as a tether lock. The anchor 400 has a webbing or frame 404 that may be compressed or folded to achieve the pre-deployment elongate configuration and that expands, on its own or manually, to achieve the deployment substantially planar configuration. The webbing or frame 404 may support a material 406, such as a fabric covering.

A tether 408 is coupled to the anchor 400. The tether 408 may be threaded through the anchor 400 through a connection point 410. A tether lumen extension 412, also referred to as a tether lumen, may be provided over the tether 408 extending between the lock 402 and the connection point 410. The tether lumen extension 412 may be flexible and may swivel around the connection point 410, or more generally around a center of the anchor 400, to aid in positioning and orientation of the anchor and generally in delivery of the anchor. A snare 414 is provided proximate the lock 402 and may be used to pull the tether 408 through the anchor 400 and lock 402 assembly.

As described above, the anchor 400 may be delivered to a target site with a catheter configured for cinching the tether 408 and activating the lock 402 to hold tension. The frame 404 is collapsible to allow loading the anchor 400 into the catheter in a collapsed elongate configuration for delivery. The frame is relatively rigid in the direction of the tether 408, or direction tension upon deployment. The material 406, or fabric covering, can aid in cushioning load or tissue and in providing a permanent fixation. It is to be appreciated, however, that in some embodiments, no material or fabric covering may be used.

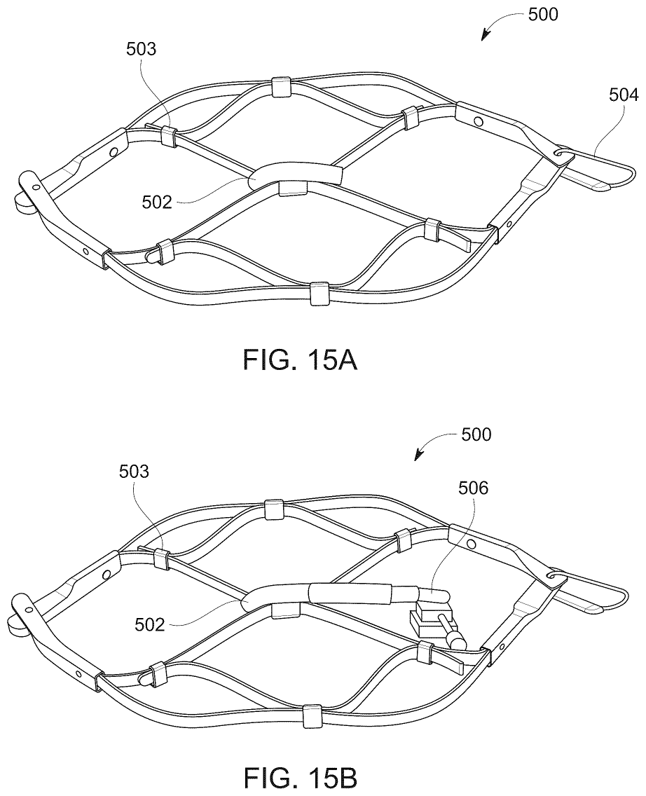

FIGS. 15a, 15b, 16a, 16b, 17a, and 17b illustrate further anchor variations and/or features. In general, the anchors comprise a deployable frame configured to expand from an elongate configuration in the anchor delivery catheter pre-deployment to a planar configuration when ejected from the anchor delivery catheter. The anchors may be self-expanding, using a shape memory material such as nitinol. Alternatively, the anchors be manually expandable using a push or pull mechanism that allows the shape and size to be controlled by a user.

The pattern of the structure of frame may vary depending on the needs of the application. In some embodiments, the frame is laser cut and thus any of a number of patterns of support may be achieved. Beams of the frame may be designed to easily flex in one direction to allow loading into a catheter but may more rigid in other direction, such as in a load or tether direction. In some embodiments, these directions may be perpendicular to each other. Alternatively, the directions may be provided at some other angle to one another.

FIGS. 15a and 15b illustrate an anchor 500 having a tether redirection feature 502. In the embodiment shown, the tether redirection feature 502 is provided generally central to the frame 503 of the anchor 500. The tether redirection feature may enhance movement of the tether within the system to facilitate smooth tightening and adjustment. This may be useful when normal access to the delivery site is limited. In the embodiment shown, the tether redirection feature aids in tensioning at an indirect angle such as at 90 degrees. FIG. 15 b illustrates a tether lock 506 that may be attached, for example at the tether redirection feature 502, pre or post delivery of the anchor. Accordingly, the tether lock 506 may be delivered in a separate step from anchor delivery or may be combined with the anchor prior to anchor delivery.

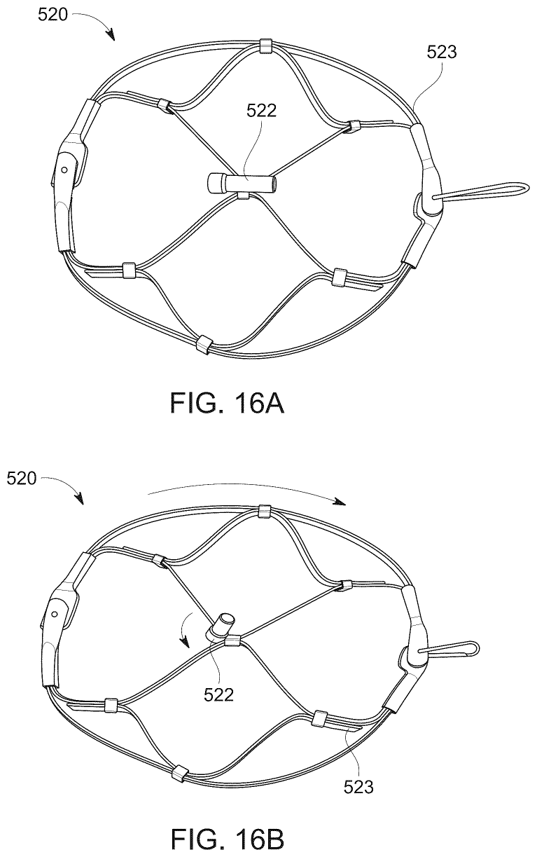

FIGS. 16a and 16b illustrate an anchor 520 having a tether redirection feature 522. In the embodiment shown, the tether redirection feature 522 is provided generally central to the frame 523 of the anchor 520. The tether redirection feature may enhance movement of the tether within the system to facilitate smooth tightening and adjustment. This may be useful when normal access to the delivery site is limited. In the embodiment shown, the tether redirection feature is a tilting/pivoting tether guide that facilitates universal orientation. FIG. 16b illustrates the tether direction feature 522 in a pivoted position;

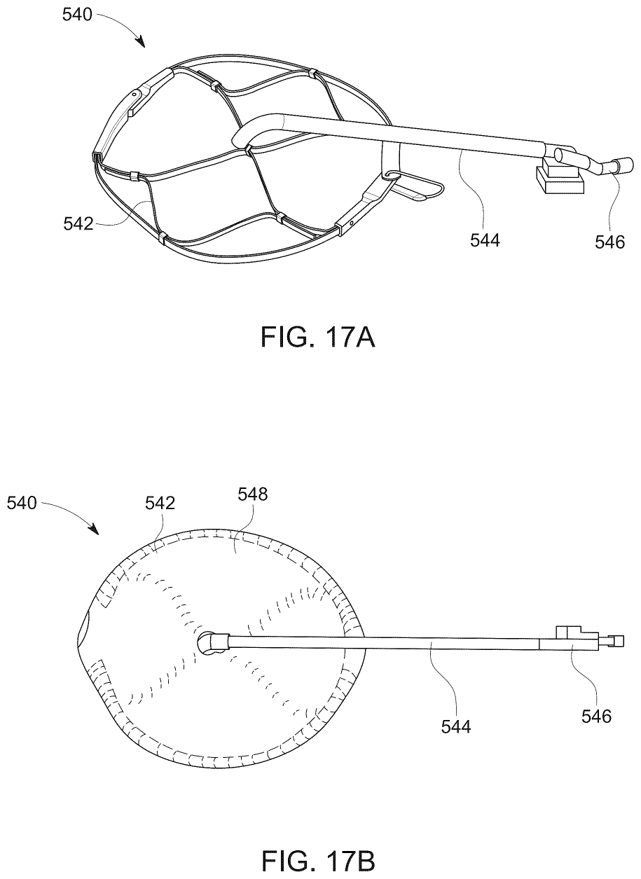

FIGS. 17a and 17b illustrate an anchor 540 in accordance with a further embodiment. As shown the anchor 540 may comprise a frame 542 having a honey-comb like support pattern. This pattern may be varied to provide more support or flexibility as desired in various applications. The anchor 540 further comprises a tether lumen extension 544 extending from a generally central position on the frame 542 to a tether lock 546. The tether lumen extension may be flexible. FIG. 17b illustrates a material covering 548 over the frame 542. The material covering 548 may be a fabric covering, a polymeric structure, or other that provided cushioning, fixation, visibility, control of ingrowth, control of attachment, or other desired feature. In some embodiments, the material covering 548 may comprise a biodegradable/resorbable material. In some embodiments, no material covering may be provided over the frame 542.

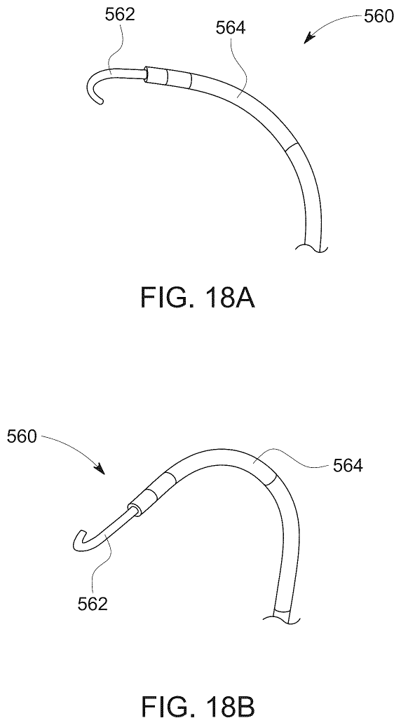

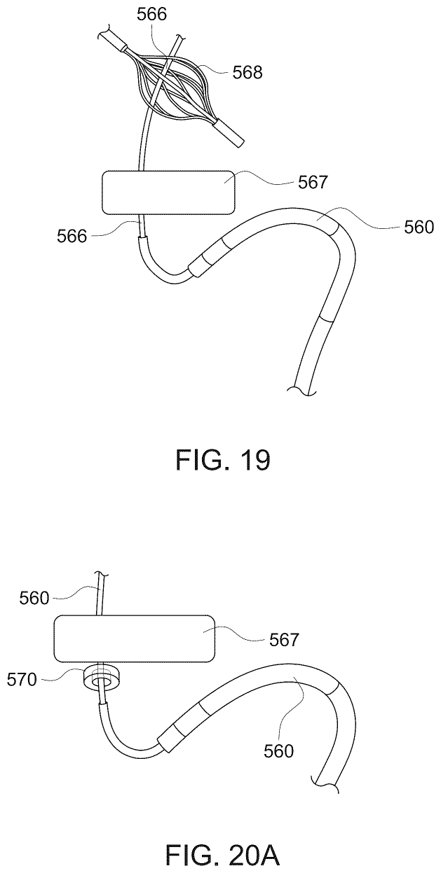

FIGS. 18a, 18b, 19a, and 19b illustrate aspects of devices for wire crossing. FIGS. 18a and 18b illustrate a wire delivery catheter 560. The wire delivery catheter 560 may include a distal catheter 562 and a proximal catheter 564. The distal catheter 562 may be an internal catheter and may be configured to deflect, rotate, advance, or retract. The proximal catheter 564 may be an external catheter and may be configured to deflect, rotate, advance, or retract. While referred to as a single unitary wire delivery catheter 560, the catheter 560 thus may be a combination of deflectable, torqueable, independent catheters that may be used to achieve a desired vector for the wire. This vector may correlate to a deployed location of an anchor, for example, the first anchor.

FIGS. 19a and 19b illustrate a wire 566 extending from the wire delivery catheter 560 through tissue 567 and to a snare 568. As the wire is advanced through the tissue 567 and captured on the opposite side of the tissue 567 with, for example, the snare 568. A suitable snare 568 is shown in FIG. 19a but other snaring mechanisms may alternatively be used. In some embodiments, RF or other energy sources may be used to aid in wire crossing. FIG. 19a illustrates a wire 566 captured by the snare 568.

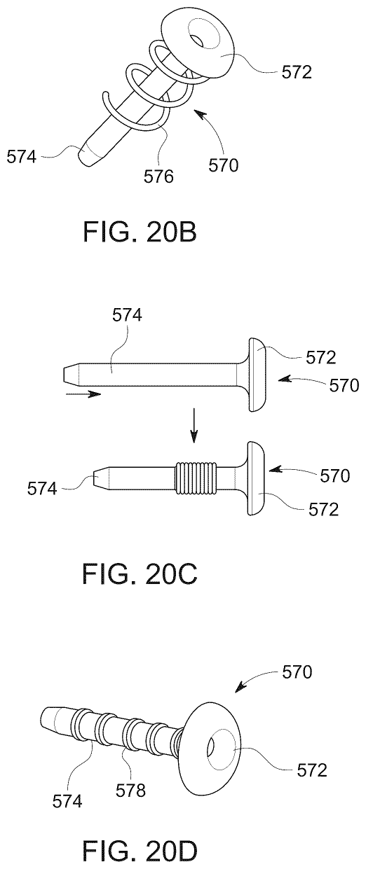

In some embodiments it may be useful to provide a protection element to protect the tissue crossing site from damage from the advancing wire and suture. FIGS. 20a, 20b, 20c, and 20d illustrate embodiments of such a protection element. FIG. 20a illustrates a wire 566 advancing from the catheter 560 to the tissue crossing site 567. A protection element 570 is provided on the proximal side of the tissue crossing site 567. The wire 566 extends through the protection element 570 and the tissue crossing site 567 to exit on the other side.

FIGS. 20b, 20c, and 20d illustrate various embodiments of a suitable protection element 570 or grommet. The tissue protection device may be provided around the wire and may be advanced before or after the wire is advanced across the tissue. The protection device aids in protection of tissue as the length of the wire and tether cross the tissue and during subsequent movement of tissue. In one embodiment, the protection element is a soft disc. In other embodiments, the protection element may coils, be threaded, be funnel shaped, be t-shaped, and/or may be collapsible. The protection element 570 may help maintain position of the anchor while threading the tether through the intended path. The protection element 570 may also help cushion the tissue from the anchor.

Each of FIGS. 20b, 20c, and 20d illustrate a protection element 570 including a disc 572 and an extension 574. In the embodiment of FIG. 20b, the extension 574 is collapsible from a fully extended position to a collapsed position. In the embodiment of FIG. 20c, the protection element 570 further includes a coil 576 provided around the extension 574. In the embodiment of FIG. 20d, the extension has ridges 578 provided therearound. In some configurations, the ridges 578 may provide a threading.

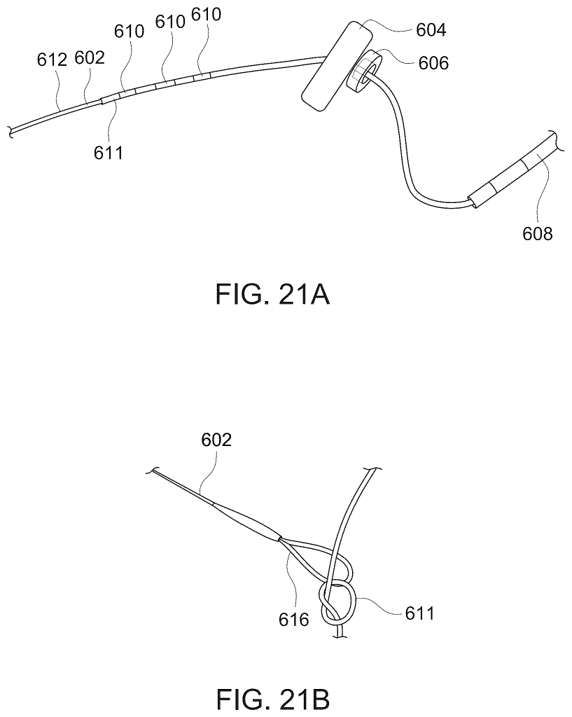

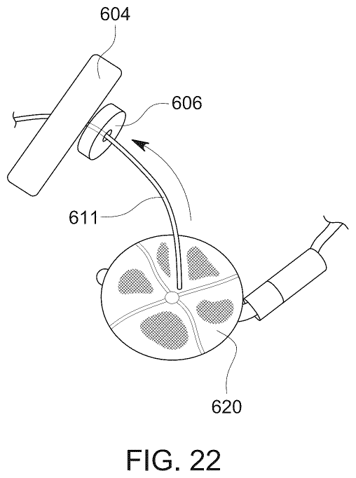

FIGS. 21a, 21b, and 22 illustrate further aspects the anchor delivery system. As shown in FIG. 21a, the wire 602, having a proximal end 612, is extended through the tissue 604 and protection element 606. An anchor delivery catheter 608 is in place on the wire 602. A tether 611 extends over the wire 602, extending through the tissue 604. A plurality of crimps 610 may be provided along the tether 611. FIG. 21b illustrates an alternative attachment mechanism wherein the tether 611 tied to an individual crimp 616 to connect the wire 602 to the tether.

The anchor delivery system facilitates secure attachment of a tether 614 to follow the crossing wire 602 through protection element 606 (optionally) and tissue 604. Once the crossing wire 602 is captured on the opposite side of the target tissue 604, the proximal end 612 of the wire may be attached to the leading end of a tether 614 (optionally radiopaque) that is or may be attached to another anchor. In some embodiments, the wire 602 may remain attached to the tether 614 for a further tissue crossing. In other embodiments, the wire 602 may be replaced for a further tissue crossing.

Returning to FIG. 21 a, a leading end of the tether 611 has multiple serial crimps 610. The distal crimps may be used first and then cut from the wire 602 and tether 611. The same or a new wire can be attached to the tether using the remaining crimps. This can be repeated for each wire that is attached or detached. The crimps 610 may comprise a metallic tube that provided holding strength to the wire 602 after crimping. The crimps 610 may further have a polymeric coating to attach and transition well with the material of the abutting tether 611.

FIG. 21b illustrates an alternative attachment mechanism of the tether 611 to the wire 602. As shown, an individual crimp-on attachment device 616 has a loop feature that allows the tying of any tether. This piece can be cut from the wire and tether and a new one attached to the same or new wire and tether.

FIG. 22 illustrates deployment of a retractable anchor 620 and tether 622. The retractable anchor 620 is deployed, and any remaining slack may be removed as the tether is drawn through the protection element 606 (optionally) and tissue 604.

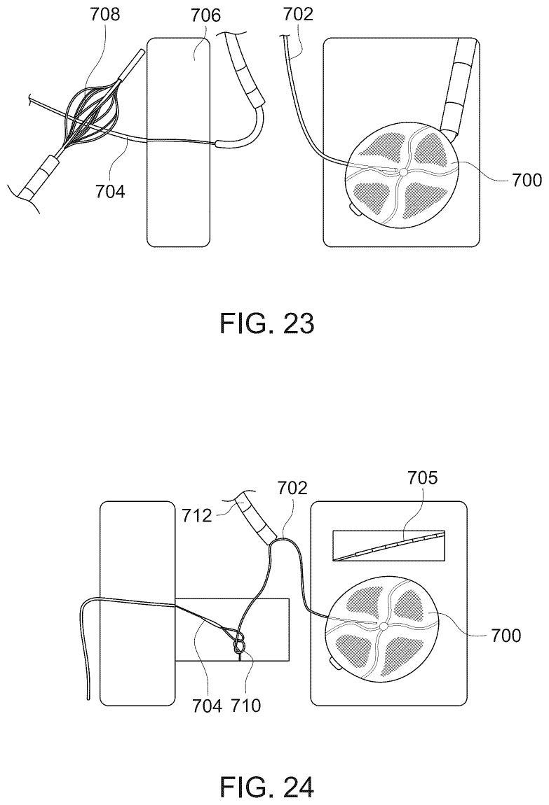

FIGS. 23, 24, and 25 illustrate aspects of a delivery system delivering a second anchor 720. FIG. 23 illustrates the first anchor 700 and tether 702 from the first crossing externalized. A second wire crossing is made using a wire crossing system such as previously described with respect to FIGS. 18a, 18b, 19a, 19b, and 20a-20d. The wire 704 crosses through tissue 706 and is captured on the opposite side of the tissue 706 and is externalized. Capturing of the wire may be with a snare 708, for example.

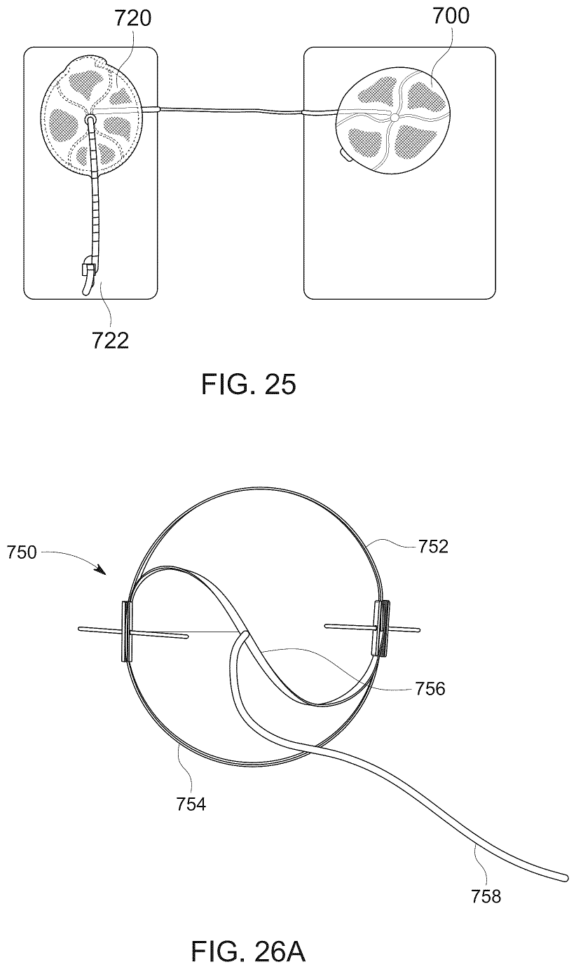

FIG. 24 illustrates the tether 702 attached to the leading wire 704. A wire/tether connection is shown at 710. This connection may be done via a loop such as shown in FIG. 21b. This attachment may be done after crossing of the wire if the wire was detached when crossing. FIG. 24 further illustrates an optional serial crimp connector 705 on the tether 702. A catheter 712 may be deployed to control the loop or slack as the wire and tether are threaded through the next layer of tissue. The anchors are deployed in a controlled manner with a proximal attachment to the anchor from the catheter, for example as shown in FIGS. 1a-1e. The anchors 700 and 720 may be previously attached to the tether 702 or may be attached via a knot or lock. FIG. 24 illustrates an anchor with a separate lock. FIG. 25 illustrates an anchor with a lock 722 attached.

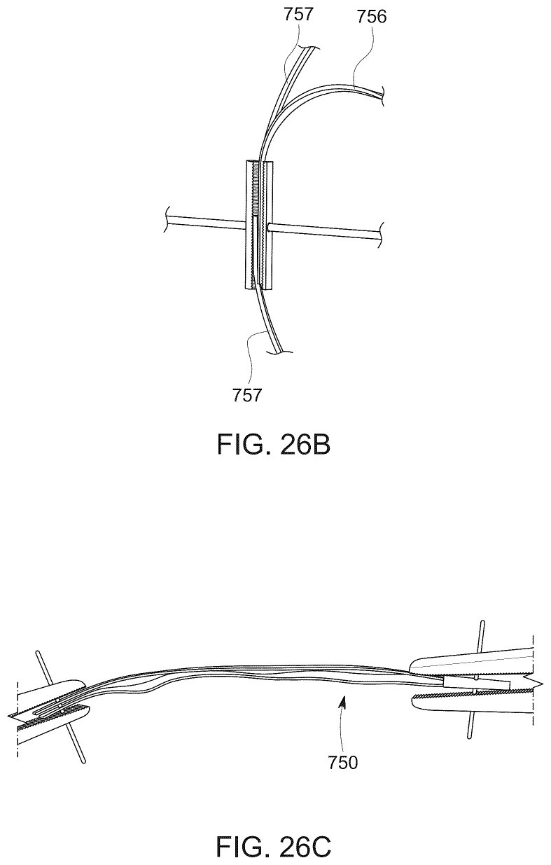

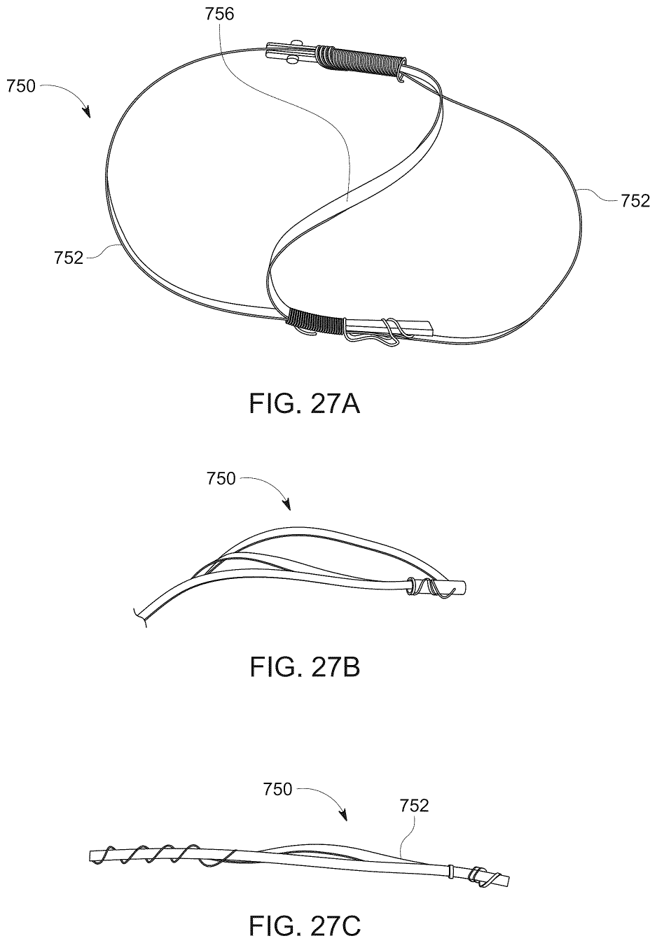

FIGS. 26a-26c and 27a-27c illustrate various perspectives of an alternative anchor embodiment. The anchor 750 is suitable for placement adjacent tissue. The anchor 750 comprises a deployable frame 752 configured to expand from an elongate configuration in an anchor delivery catheter pre-deployment to a planar configuration when ejected from the anchor delivery catheter. The frame 752 comprises an outer ring or peripheral frame 754 and an center member or central support 756. The pieces of the frame 752 may be formed of a memory shape material such as nitinol. In one embodiment, the outer ring 754 comprises opposing rib members 757. In one embodiment, the center member is a center spiral spring. The center member 756 may be connected to opposing rib members 757 on each side, shown in FIG. 26b. This achieves connection and also functions to drive the opposing rib member 757 apart to form the outer ring 754. A tether 758 may be attached to the center member 756. FIG. 26c illustrates the anchor 750 in a flattened elongate configuration. As shown, in this embodiment, each of the opposing rib member 757 and center member 756 have similar or the same lengths.

The embodiment of FIGS. 27a-27c differs from the embodiment of FIGS. 26a-26c in connection of the center member 756 to the opposing rib members 757. FIG. 27a illustrates the frame 752 in an expanded planar configuration. FIG. 27b illustrates the frame 752 in a partially collapsed configuration. FIG. 27c illustrate the frame 752 in an elongate configuration.

Accordingly, systems and methods for delivering tensioning elements, such as tethers, and deployable anchors, to a desired location within the anatomy of a patient are described. More specifically, a system and method for delivering deployable anchors to a desire location within the anatomy and tethering the anchors together through tissue is disclosed. The deployable anchors may comprise anchors that have a pre-deployment elongate configuration and a post-deployment planar configuration. In the post-deployment planar configuration, the anchors act to distribute force over a surface area of the tissue. Such anchors and tethers may be useful in structural heart applications but may also be used in an area where compression, reshaping, or movement of an organ or luminal structure is desired.

While the present disclosure is directed to the delivery of such anchors and tethers in structural heart applications, the disclosed embodiments can be used for other applications such as compression of the prostate or movement of luminal structures and the like. The disclosed embodiments can be used within any organ or luminal structure that needs to be reshaped or where a portion of the organ or luminal structure needs to be moved temporarily or permanently. Thus, the disclosed embodiments are meant to be illustrative only.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

1-30. (canceled)

31. A method for deploying an anchor at a location within a body of a patient, the method comprising:
providing an anchor delivery catheter defining a longitudinal central axis along its length, and a lateral direction arranged orthogonally with respect to the longitudinal central axis, the anchor delivery catheter including a deployable anchor disposed within a sheath at a distal end region of the anchor delivery catheter, the anchor including:
a laterally expandable frame formed from a framework of strands of shape memory material, the laterally expandable frame having:
a laterally collapsed configuration wherein the strands of shape memory material are compressed along the lateral direction to form a laterally-compressed elongate body having a proximal end and a distal end; and
a laterally expanded configuration wherein the strands of shape memory material are expanded along the lateral direction to form a planar area defined by a central region and a peripheral region, wherein:
strands of the shape memory material defining the central region are mechanically connected to strands of the shape memory material defining the peripheral region to form a continuous web of strands of the shape memory material;
the frame has a proximally-facing edge formed by the lateral expansion of the proximal end of the laterally-compressed elongate body; and
the frame has a distally-facing edge formed by the lateral expansion of the distal end of the laterally-compressed elongate body;
a tether mount mechanically coupled to the strands of shape memory material in the central region of the frame; and
a tether attached to and extending away from the tether mount along a proximal direction through the sheath and through a lumen of the anchor delivery catheter;
introducing the distal end of the catheter to an anatomical location;
advancing the distal end of the laterally-compressed elongate body out of the sheath along a distal direction;
permitting the frame of the anchor to expand along the lateral direction to permit the distal end of the laterally-compressed elongate body to expand along the lateral direction to form the distally-facing edge of the frame;
advancing the proximal end of the laterally-compressed elongate body out of the sheath along the distal direction;
permitting the frame of the anchor to further expand along the lateral direction to permit the proximal end of the laterally-compressed elongate body to expand along the lateral direction to form the distally-facing edge of the frame, wherein the planar area of the frame intersects the central axis of the catheter upon being deployed from the sheath;
reorienting the frame of the anchor to that the planar area of the frame lays in a plane that is orthogonal to the central axis of the catheter; and
applying tension to the tether to pull the anchor against an anatomical surface.

32. The method of claim 31, wherein the tether mount is pivotally mounted to the frame by way of a pivot, and further wherein the method further comprises rotating the tether mount about the pivot to apply tension to the anchor.

33. The method of claim 31, wherein tension is applied to the tether along a direction that is orthogonal with respect to the planar area of the frame.

34. The method of claim 31, wherein tension is applied to the tether along a direction that is oblique with respect to the planar area of the frame.

35. The method of claim 31, wherein tension is applied to the tether along a direction that is parallel with respect to the planar area of the frame.

36. The method of claim 31, wherein the anchor further includes a membrane layer covering at least a portion of the frame.

37. The method of claim 36, wherein the membrane layer includes a fabric covering.

38. The method of claim 31, wherein the planar area of the frame comprises a flat plane.

39. A system for deploying an anchor at a location within a body of a patient, the system comprising:
an anchor delivery catheter defining a longitudinal central axis along its length, and a lateral direction arranged orthogonally with respect to the longitudinal central axis, the anchor delivery catheter including a deployable anchor disposed within a sheath at a distal end region of the anchor delivery catheter, the anchor including:
a laterally expandable frame formed from a framework of strands of shape memory material, the laterally expandable frame having:
a laterally collapsed configuration wherein the strands of shape memory material are compressed along the lateral direction to form a laterally-compressed elongate body having a proximal end and a distal end; and
a laterally expanded configuration wherein the strands of shape memory material are expanded along the lateral direction to form a planar area defined by a central region and a peripheral region, wherein:
strands of the shape memory material defining the central region are mechanically connected to strands of the shape memory material defining the peripheral region to form a continuous web of strands of the shape memory material;
the frame has a proximally-facing edge formed by the lateral expansion of the proximal end of the laterally-compressed elongate body; and
the frame has a distally-facing edge formed by the lateral expansion of the distal end of the laterally-compressed elongate body.

40. The system of claim 39; wherein the frame further includes a tether mount mechanically coupled to the strands of shape memory material in the central region of the frame.

41. The system of claim 40; wherein the frame further includes a tether attached to and extending away from the tether mount along a proximal direction through the sheath and through a lumen of the anchor delivery catheter.

42. The system of claim 41, wherein the tether mount is pivotally mounted to the frame by way of a pivot.

43. The system of claim 39, wherein the anchor further includes a membrane layer covering at least a portion of the frame.

44. The system of claim 43, wherein the membrane layer includes a fabric covering.

45. The system of claim 39, wherein the planar area of the frame comprises a flat plane.
